(19) United States
(12) Patent Application Publication
(10) Pub. No.: US 2010/0322859 A1
(43) Pub. Date: Dec. 23, 2010

(54) ORAL DRUG COMPLIANCE MONITORING USING MAGNETIC-FIELD SENSORS

(75) Inventors: Christopher M. Jones, Midland, MI (US); Maysam Ghovanloo, Atlanta, GA (US); Douglas P. White, Midland, MI (US); Peter K. Mercure, Midland, MI (US); Malcolm W. Warren, II, Sanford, MI (US)

Correspondence Address: The Dow Chemical Company/ Butzel Long, Stoneridge West 41000 Woodward Ave, Bloomfield Hills, MI, 48304, US

(73) Assignees: DOW GLOBAL TECHNOLOGIES INC., Midland, MI (US); NORTH CAROLINA STATE UNIVERSITY, Raleigh, NC (US)

(21) Appl. No.: 12/297,321

(22) PCT Filed: Apr. 24, 2007
(86) PCT No.: PCT/US07/10154

§ 371 Date: Nov. 13, 2009

Related U.S. Application Data
(60) Provisional application No. 60/794,638, filed on Apr. 25, 2006.

(51) Int. Cl. A61K 49/00; G01R 33/12; G01N 33/15

(52) U.S. Cl. 424/9.1; 324/228

(57) ABSTRACT

A method to measure compliance with a pharmaceutical regimen, by the steps of: (a) ingesting a dose of a medication (10) into the gastrointestinal tract of a person, the dose of medication (10) comprising a drug formulation (13) and a permanent magnet (14); (b) as a result of the ingestion of step (a), detecting passage of the permanent magnet (14) past at least two magnetic field sensors (20) positioned apart from each other and adjacent to the gastrointestinal tract; and (c) measuring compliance with the pharmaceutical regimen by way of the detection of step (b). And, apparatus useful to measure compliance with a pharmaceutical regimen by detecting ingestion of a dose of medication (10) comprising a drug formulation (13) and a permanent magnet (14), the apparatus including at least two magnetic field sensors (20) positioned apart from each other on a necklace (22), each magnetic field sensor (20) being in electronic communication with a microprocessor for receiving signals from the magnetic field sensors (20) to determine the passage of a permanent magnet (14) between the magnetic field sensors (20).

This application claims the benefit of U.S. Provisional Patent application Ser. No. 60/794,638 filed Apr. 25, 2006. The instant invention relates to oral drug compliance monitoring, and, more particularly, to a means for the detection of ingestion of a drug formulation using magnetic-field sensors.

### BACKGROUND OF THE INVENTION

Non-compliance of patients to drug regimens prescribed by their physician results in increased cost of medical care, higher complication rates, as well as drug wastage. Non-compliance refers to the failure to take the prescribed dosage at the prescribed time which results in undermedication or overmedication. In a survey of 57 non-compliance studies, non-compliance ranged from 15% to as high as 95% in all study populations, regardless of medications, patient population characteristics, drug being delivered or study methodology [Greenberg R N: Overview of patient compliance with medication dosing: A literature review. Clinical Therapeutics, 6(5):592-599, 1984].

In the clinical drug stage, accurately measuring compliance can lead to benefits such as: improved statistical reliability of a clinical study; clinical studies being completed sooner; and a determination of the effect of non-compliance as a function of the degree of non-compliance. In the therapeutic stage, accurately measuring compliance has a number of important benefits such as: warning a patient about the potential for developing a drug resistant infection related to poor compliance; and identifying a side effect of a drug related to overdosing.

Confirmation of drug compliance by way of direct observation by trained persons is effective but impractical in most situations. Confirmation of drug compliance by blood or urine analysis is also impractical in most situations. Transdermal detection devices attached to the skin of a patient have been developed which detect ingested drug components through the skin and such devices can transmit a signal to a remote receiver at an external site such as a healthcare facility, see U.S. Pat. No. 6,663,846 and USPAP 2005/0031536. Electronic sensor systems have been developed which detect ingested drug components in the breath of a patient, see USPAP 2004/0081587. Radio frequency identification (RFID) tags have been incorporated into drug pills, each tag capable of identifying the type of medication, its dosage, and its lot number by way of a unique code emitted by the tag when interrogated by a corresponding radio frequency “reader”, see U.S. Pat. No. 6,366,206. The RFID of the '206 patent can incorporate a biosensor that detects moisture or change in pH to determine whether the pill has dissolved and exposed the RFID tag to the environment of the gastrointestinal system. The technology of the '206 patent requires a highly specialized spherical RFID semiconductor and requires that the semiconductor also include the sensor that detects moisture or change in pH to determine whether the pill has dissolved and exposed the RFID tag to the environment of the gastrointestinal system. More generally, RFID tags are relatively expensive to incorporate into a medication pill or capsule. And, if the RFID tag is an “active” RFID tag (i.e., an RFID tag that incorporates a power source, usually a battery), then there may be additional safety and regulatory requirements.

U.S. Pat. No. 5,079,006 describes the use of a pharmaceutical preparation for oral or anal administration containing, in addition to a selected pharmacologically active agent, at least one magnetic material capable of reacting to an externally acting magnetic field of an electronic monitoring device, so that after administration to a patient, a signal is produced that is selectively detectable by such a monitoring device, thus providing information on whether the preparation has been taken or not by the patient and/or facilitates locating the preparation or the magnetic material in the region of the gastrointestinal tract of the patient. USPAP 20040059204 discloses a drug capsule having a magnetic member, wherein the magnetic member permits manipulation of the capsule from a non-body location. U.S. Pat. No. 6,374,670 describes a method and apparatus for monitoring of gut motility using an ingestible magnet which is swallowed by the patient to permit linear and rotational movement detected by an external compass. U.S. Pat. No. 6,440,069 also represents an advance in the art. Despite the advances in the prior art, there remains a need for improved methods for oral drug compliance monitoring.

### SUMMARY OF THE INVENTION

The instant invention is a solution to the above stated problem. More specifically, the instant invention is a method to measure compliance with a pharmaceutical regimen, comprising the steps of: (a) ingesting a dose of a medication into the gastrointestinal tract of a person, the dose of medication comprising a drug formulation and a permanent magnet; (b) as a result of the ingestion of step (a), detecting passage of the permanent magnet past at least two magnetic field sensors positioned apart from each other and adjacent to the gastrointestinal tract; and (c) measuring compliance with the pharmaceutical regimen by way of the detection of step (b).

In another embodiment, the instant invention is an apparatus useful to measure compliance with a pharmaceutical regimen by detecting ingestion of a dose of medication comprising a drug formulation and a permanent magnet, comprising: at least two magnetic field sensors positioned apart from each other on a necklace, each magnetic field sensor being in electronic communication with a microprocessor for receiving signals from the magnetic field sensors to determine the passage of a permanent magnet between the magnetic field sensors.

### BRIEF DESCRIPTION OF THE DRAWINGS

FIG. 1 is an enlarged side view, part in cross section and part in full, of a dose of medication comprising a permanent magnet according to a preferred embodiment of the instant invention;

FIG. 2 is a side view in full of a permanent magnet coated with a relatively thick coating wherein the surface of the coating has the shape of an ellipsoid according to a preferred embodiment of the instant invention;

FIG. 3 is an illustration, part in full and part broken away, of a patient wearing magnetic field sensors;

FIG. 4 is a cross-sectional view of a neck of a patient around which is positioned a necklace incorporating magnetic field sensors according to a preferred embodiment of the instant invention;

FIG. 5 is a side view of a neck of a patient around which is positioned a necklace incorporating magnetic field sensors according to a preferred embodiment of the instant invention;

FIG. 6 is an overview of the software flow of a preferred embodiment of the instant invention;

FIG. 7 shows the magnetic field sensor output waveforms when a permanent magnet passed inside the necklace of FIG. 5; and

FIG. 8 is a flowchart for a preferred algorithm for determining when a patient ingests the dose of medication shown in FIG. 1, the algorithm based on the sensor output amplitude values using the necklace of FIG. 5.

### DETAILED DESCRIPTION

The instant invention provides a method for clinical trial data analysis, and for physicians and their patients, to have an objective measurement of when and in what dose medication was ingested by a patient. The dose device used in the instant invention comprises a permanent magnet which is relatively inexpensive, inherently wireless, requires no power, can be compact in size and safe to use, and has an unlimited shelf life. The method of the instant invention provides accurate, reliable and robust detection, is non invasive, sensitive, convenient and difficult to deceive. More specifically, the instant invention is a method to measure compliance with a pharmaceutical regimen, comprising the steps of (a) ingesting a dose of a medication into the gastrointestinal tract of a person, the dose of medication comprising a drug formulation and a permanent magnet; (b) as a result of the ingestion of step (a), detecting passage of the permanent magnet past at least two magnetic field sensors positioned apart from each other and adjacent to the gastrointestinal tract; and (c) measuring compliance with the pharmaceutical regimen by way of the detection of step (b). Preferably, step (b) comprises the generation of a time and date stamped data point which is stored for use in step (c). Preferably, the time and date stamped data point is stored in digital form in, for example and without limitation thereto, a personal data assistant device or a personal computer. The use of a personal data assistant device in the instant invention provides a low cost and widely available technology that is light weight, can be programmed to remind a patient to take a medication, can readily transfer stored data points to, for example and without limitation thereto, a digital computer via wire or wireless communication. In another embodiment, the instant invention is an apparatus useful to measure compliance with a pharmaceutical regimen by detecting ingestion of a dose of medication comprising a drug formulation and a permanent magnet, comprising: at least two magnetic field sensors positioned apart from each other on a necklace, each magnetic field sensor being in electronic communication with a microprocessor for receiving signals from the magnetic field sensors to determine the passage of a permanent magnet between the magnetic field sensors. Preferably, the apparatus comprises three magnetic field sensors positioned apart from each other incorporated into the necklace. Most preferably, such three magnetic field sensors are positioned about equidistant from each other. The apparatus can further comprise a digital personal data assistant (PDA) configured to receive a time and date stamped data point transmitted by a radio frequency transmitter in electrical communication with the microprocessor.

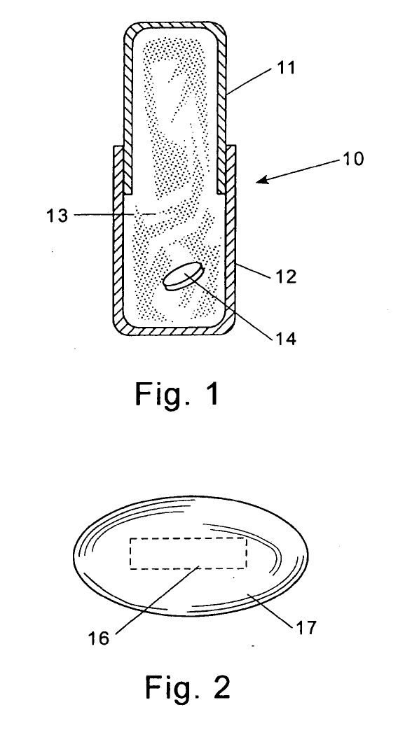

Referring now to FIG. 1, therein is shown a medication or drug dose 10 according to the instant invention, consisting of an upper gelatin capsule portion 11, lower gelatin capsule portion 12 containing a drug formulation 13 and a disk shaped rare earth permanent magnet 14. Of course, a drug dose of the instant invention can be in any form such as, without limitation thereto, a pill or tablet. The magnet 14 is preferably coated with silicone polymer (or other suitable coating) so that the magnet 14 is not exposed to the fluids of the gastrointestinal tract. Any suitable permanent magnet can be used such as RadioShack permanent magnet part number 64-1895.

Referring now to FIG. 2 is a side view in full of a permanent magnet coated with a relatively thick coating wherein the surface of the coating has the shape of an ellipsoid according to a preferred embodiment of the instant invention for incorporation with a dose of medication according to the instant invention.

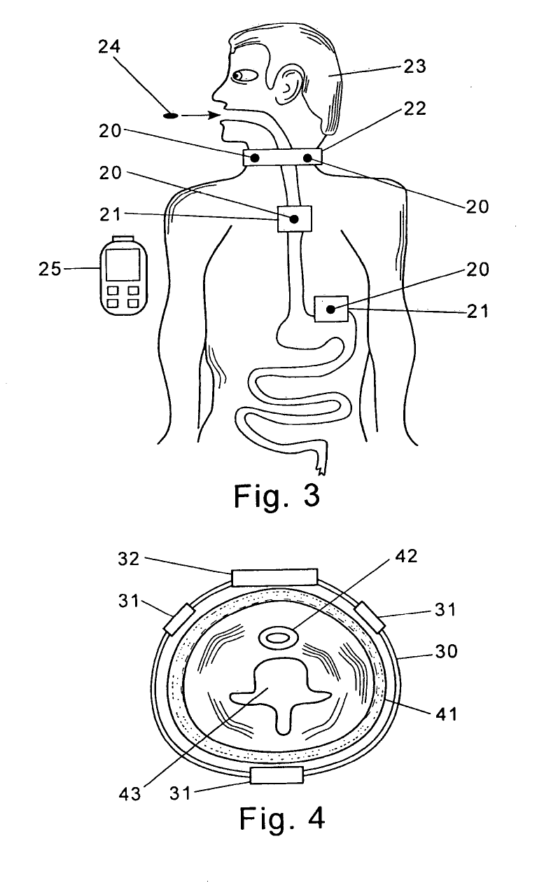

Referring now to FIG. 3, therein is shown an illustration, part in full and part broken away, of a patient 23 wearing magnetic field sensors 20 incorporated into patches 21 or a necklace 22. The magnetic-field sensor based wireless drug compliance monitoring (DCM) system is preferably composed of three major components: the magnetic tracer 24, detection device (i.e., the sensors 20), and data delivery device such a digital personal data assistant (PDA) 25.

The tracer is a tiny disk shaped permanent magnet preferably coated with silicone or other polymer based bio-compatible materials. The tracer generates the magnetic field that penetrates through tissue and provides the detector with necessary information about the movement of the dose of medication as it passes through the esophagus.

The detection device is preferably a wearable high resolution magnetic-field sensor array that is located around the neck in different orientations. Such an array can detect the tracer regardless of its orientation when it passes through the esophagus. The sensors are preferably followed by the control unit that consists of a power source (battery), a low power microcontroller (i.e., a microprocessor), and an RF wireless transceiver. The signal processing routine that runs on the microcontroller detects a “dose ingestion event” by continuously looking at multiple sensor outputs and then wirelessly transmits occurrence of the event to the data delivery device.

The data delivery device, which preferably takes advantage of the available portable computing technology (PDA), is also wearable and equipped with a dedicated RF transceiver, tuned to the detector transceiver. Upon reception of a “dose ingestion event”, the data delivery device sends an acknowledgment (ACK) to the detector and time/date stamps the event. All the received events are stored in the PDA memory for later retrieval by, for example, a physician.

The magnetic DCM system is non-invasive, inherently wireless, transparent to all, kinds of tissue, and safe. The wearable monitoring necklace and PDA have no chemical effect on the patient's body and can be made to be almost unobtrusive. The magnetic tracers are preferably coated by biocompatible materials. Several strong magnets in the gastrointestinal tract can result in blockage. As a result, the magnetic-field sensors should be sensitive enough to detect the changes in the magnetic field resulted from small and relatively weak magnets. These changes are comparable to the earth magnetic field and the DCM signal processing algorithm should be able to discriminate between the two fields. Therefore, it is necessary to use multiple sensors in different directions. With a combination of multiple sensors, the pattern of magnetic field variations that result from passage of a dose of medication according to the instant invention through the esophagus will also be distinguishable from other patterns that result, for example, from electromagnetic interference or passing such a dose outside the necklace in order to eliminate any intentional or accidental errors. It is also possible to sensitize the system to two or more specific magnets with certain magnetic strength in order to detect and monitor two or more drugs that are consumed together.

The detector necklace power consumption should preferably be small enough such that a small battery will last for a long period of time, preferably from one doctor's visit to the next. The wireless transceiver is the most power consuming block on the detector. However, since the “dose ingestion event” is only triggered perhaps a few times per day, the system operates at a very low-power monitoring mode except for the relatively short time needed to transmit a dose ingestion event. Further, the power management block preferably scans the magnetic-field sensors and turns only one of them on at a time, resulting in additional power savings.

A permanent magnet functions as the tracer. It takes about 24 hours on average for each tracer to pass through the GI tract. Therefore, for medications that need to be taken more than once a day, there is a possibility that two or more magnets will exist inside the GI tract at the same time. Serious conditions such as GI tract obstruction might possibly arise if two or more magnets are stuck together across different turns of the GI tract. Therefore, the strength of the magnetic tracer should be sufficiently low to prevent such occurrence. Another important factor is the thickness of the preferred polymer coating around each magnet especially around the two poles, which will eliminate two adjacent magnets from getting closer than a certain distance. FIG. 2 shows a permanent magnet 16 having a relatively thick coating 17, especially around the poles of the magnet 16. Fortunately the force that the magnets exert on one another rapidly decays with their distance. In addition, the magnetic field of the magnetic tracer should preferably be stable over time and temperature.

A preferred tracer is a cylindrical rare earth permanent magnet from RadioShack (Catalog No. 64-1895) or more preferably a weaker magnet of the same size manufactured by Magnetix.

Performance, power consumption, and size are the three important parameters in the system of the instant invention. To detect the relatively weak magnetic field generated by a tiny permanent magnet embedded in, for example a pill, tablet or capsule, high resolution magnetic-field sensors are preferred. It takes perhaps 1-3 seconds for the pill, tablet or capsule to pass through the esophagus and this is the only time when the tracer is in the vicinity of the detector device. Therefore, the sampling frequency must be sufficiently high to provide enough samples for signal processing routines to obtain effective detection of the “dose ingestion event”. On the other hand, a higher sampling rate requires faster hardware and more power consumption. There is also a direct relationship between the delay and resolution of the magnetic-field sensors, i.e. higher resolution samples require more time to be taken. Therefore, there should preferably be a balance between the sensitivity, speed, and power consumption of the detector device.

The most popular magnetic-field sensors available in the market are Hall Effect, magneto-resistive and magneto-inductive sensors. The Magneto-inductive sensor is preferred in the instant invention. A preferred sensor for use in the instant invention is a Micromag2 sensor module available from PNI Corporation (CA USA). The Micromag2 is an integrated 2-axis magnetic field sensing module manufactured by the PNI Corporation which incorporates a temperature and noise stabilized oscillator/counter circuit with a serial peripheral interface (SPI) bus. The microprocessor compatible SPI interface allows ready access to the Micromag2 measurement parameters and resulting field measurement data.

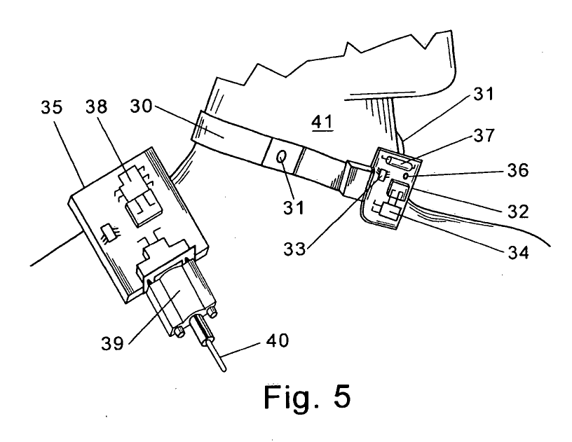

FIGS. 4 and 5 show a necklace incorporating three such Micromag2 magneto-inductive sensor modules 31 mounted on a flat ribbon cable 30 to form the necklace and capture the magnetic field variation around the user's neck 41. FIG. 4 additionally shows the esophagus 42 and vertebra 43. The sensor modules 31 are equally distanced on the flat cable 30 which forms a common bus between modules and the control unit 32. The control unit 32 is preferably made to be much smaller than shown in FIG. 5 and can even be ornamented with jewelry to improve its appearance. Each sensor module contains two perpendicular low power magneto-inductive sensors with the highest resolution of 150 μGauss, measuring the magnetic strength in two directions, vertical (Z) and horizontal (X). If the necklace forms a perfect circle around the neck, the horizontal sensor axes make an equilateral triangle and the vertical sensors axes will be parallel to the tracer path. Since it takes perhaps 1-3 second for the tracer to pass through the neck area, a scan rate of 10 Hz is found to be sufficient to detect an ingestion event.

A low power Atmega 32L microprocessor (available from Atmel Corporation, USA) reads the digital outputs of the three Micromag2 sensors via serial port interface (SPI). All the measurement results are processed within the microprocessor and used to generate the ingestion event. The microprocessor also controls a commercial RF transceiver (LAIPAC Corporation, USA, TRF-2.4G) operating at 2.4˜2.5 GHz in the industrial-scientific-medical (ISM) band. The transceiver 34 is first configured as a transmitter to transmit the ingestion event to the receiver 35 of the data delivery device and then switches to receive mode to receive the acknowledgement (“ACK”) signal. The “data delivery device” is comprised of the receiver 35 and the PDA (not shown). Preferably, the receiver 35 is combined with a PDA. In the absence of an ACK, the microprocessor preferably repeats the event transmission four more times and notifies the user by turning on a red LED 36 if it does not receive any response back. The control unit 32 is powered by a 3.3 V battery 37.

An RF transceiver 38 (LAIPAC Corporation, USA, TRF-2.4G) is configured as a receiver on the data delivery device 35 to capture the event detection message transmitted by detection device 32. In the configuration shown, the receiver 35 communicates the event through RS-232 port 39 to a PC or PDA via cable 40. The PC or PDA, runs a DCM graphical user interface (GUI) in the LabVIEW environment. The data delivery device then confirms reception of the event by sending an ACK message. The transmission distance for LAIPAC 2.4G is up to 200 m, which allows the user to place, for example, a PDA at a convenient location around, for example, the person's house.

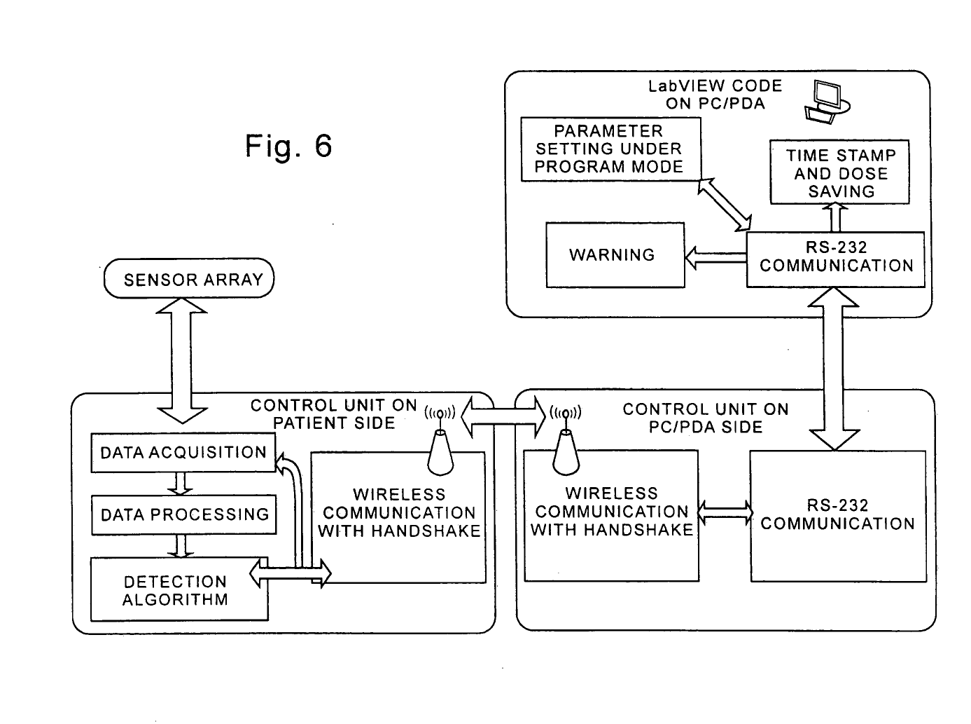

Software in the preferred DCM system is divided into three parts as depicted in FIG. 6: microcontroller code for the control unit on the necklace detector, microcontroller code on the PC/PDA transceiver board, and the LabVIEW code running on PC/PDA.

The Detection Algorithm for the control unit of the necklace detector, Wireless Handshaking Protocol for data flow and the LabVIEW GUI running on PC/PDA will now be discussed.

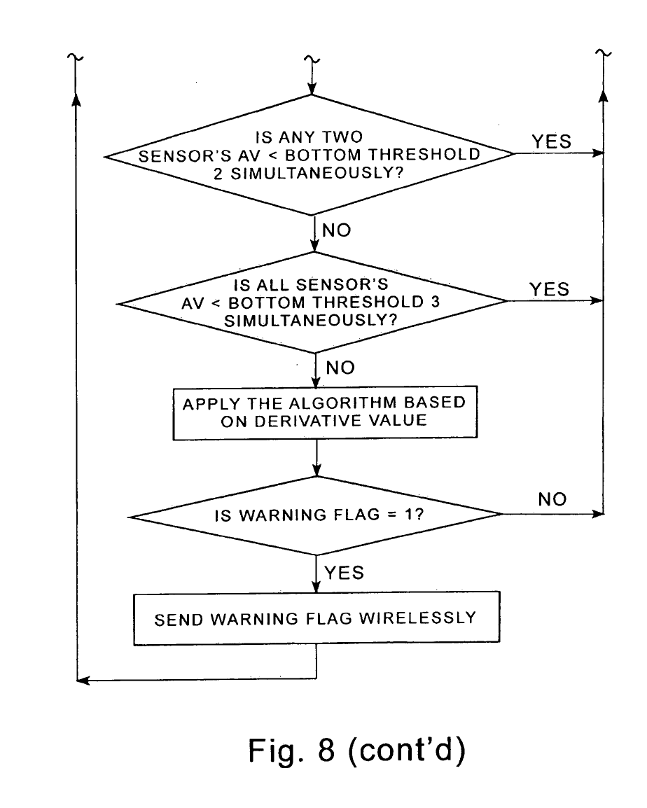

The microcontroller has limited calculation and data processing capacity relative to a PC. Therefore, the algorithm running on the microcontroller is preferably simple and small but providing efficient calculations. In the preferred embodiment, we have implemented an efficient Multi-Threshold based Algorithm outlined in FIG. 8 to provide an optimum between the need for accuracy and the need to control computational complexity.

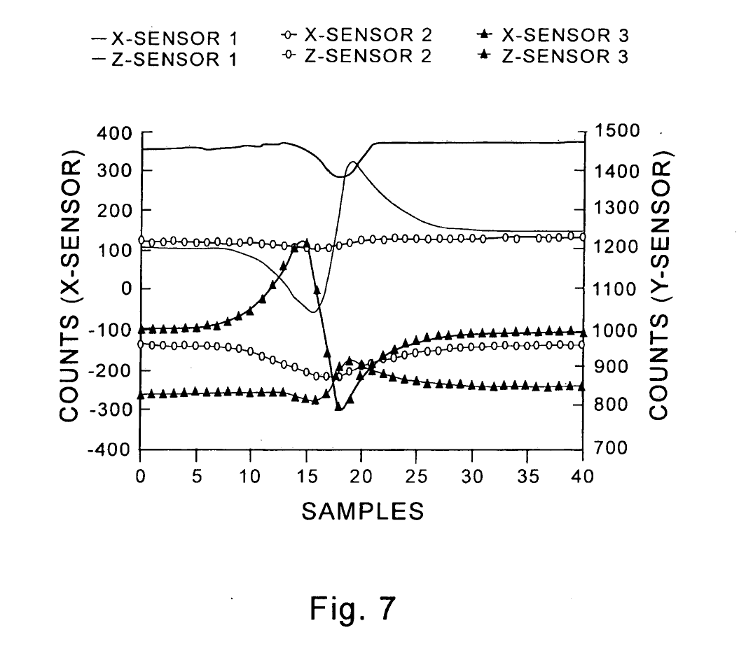

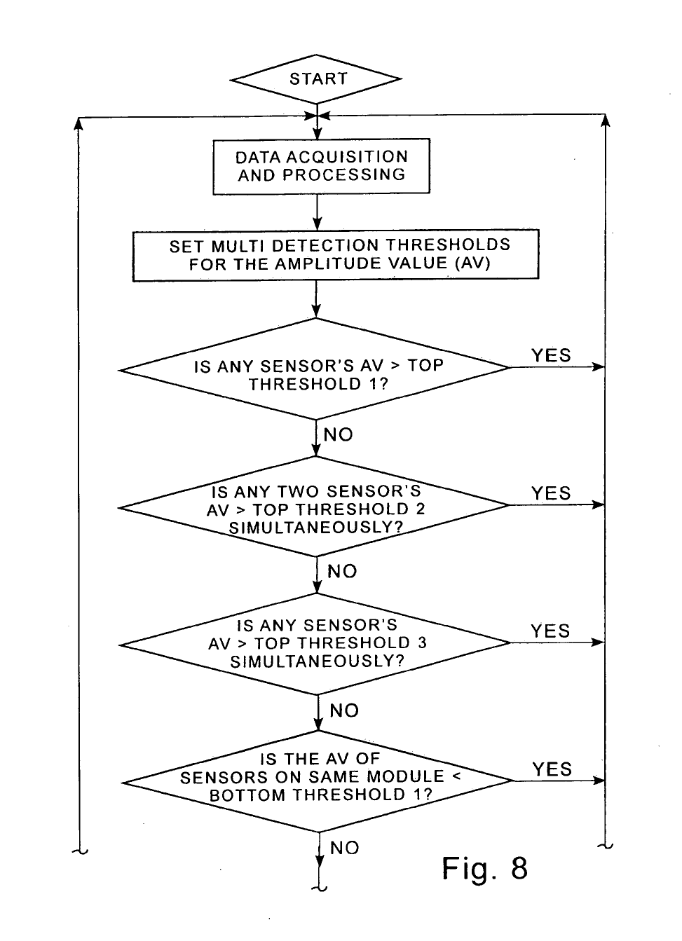

The overall gist of the algorithm is to look at the individual and combination of outputs of sensors (shown, for example in FIG. 7) and compare them to different thresholds. These thresholds depend on the type and strength of the magnet used as the tracer and can be preset by calibration experiments. Only when all threshold requirements are met, will the system conclude that patient has taken a pill or the like and generate a “dose ingestion event”. Otherwise, the case will be rejected by the algorithm.

The thresholds are preferably set relative to the sensors average output and indicate the maximum and minimum amplitude values (AV) allowed for single or multiple sensors when the tracer is passing inside the detector and not too close to the sensors. Since no certain horizontal position for the necklace is specified, i.e. the necklace can rotate around the neck, all sensor modules are considered identical and distributed evenly on the necklace. Therefore, all horizontal (X) and all vertical (Z) sensor thresholds are assumed to be the same.

Thresholds for individual sensors: Since it is impossible for the tracer magnet to move against the sensor in a real ingestion event, there is maximum AV for each sensor when the magnet is passing through the esophagus regardless of the orientation of the magnet. Therefore, upper thresholds for individual sensors are set up to indicate the maximum acceptable response when the magnet is closest to the sensor in the esophagus and parallel to the measurement axis of the sensor. If any sensor exceeds such a threshold, the magnet is considered to be outside the necklace. There is no minimum threshold for individual sensors because a sensor can give zero output when the magnet is facing the sensors and the magnetic flux is perpendicular to the sensor axis.
Thresholds for multiple sensors with the same orientation on different modules: Since the relative position and orientation of different modules is almost fixed, the outputs of all X (or Z) sensors are related. Meanwhile, the magnet cannot be very close to or far from two or three sensors (same direction but in different modules) at the same time when it is passing inside the necklace in a real ingestion event. Therefore, there are maximum and minimum values for multi sensors' simultaneous output. These maximum and minimum values are set as the upper and lower thresholds for every two or all three sensors of the same type (X or Z). By adjusting the position and orientation of the tracer when it is passing inside the necklace, these thresholds can be measured and used to detect the tracer during normal operation of the system.
Thresholds for dual sensors with different orientation on the same module: The outputs of the X and Z sensors on the same module are related due to their constant relative positions. The maximum AV of both sensors cannot be very small simultaneously, when the magnet is passing from inside the necklace in a real ingestion event. Therefore, the lower thresholds are defined for simultaneous output signals generated by every module. If X and Z sensors on the same module are both below this lower threshold level, the magnet is considered to be outside the necklace.
Additional Thresholds: The relative amplitude thresholds can reject the effects of permanent magnets that are not in the right position or do not have the expected strength. However, they cannot sufficiently reject the signal variations resulted from the earth magnetic field when the patient moves or rotates. Therefore, additional constraints are also preferably used in the signal processing algorithm based on a derivative value (DV).

By comparing the derivative waveforms between a normal magnet passing through the necklace and patient's rotation, two significant differences are observed. First, the maximum values of dX/dt (or dZ/dt) are almost the same among all modules when the patient rotates 360 degrees. However, those are ordinarily quite different from each other when the magnet is passing inside the esophagus when the field generated by the tracer magnet is relatively weak such that only the sensor closest to magnet can give significant change in its response, while the earth magnetic field can affect all the sensors. Second, the time between maximum and minimum values of the derivative waveforms (ÄT) is also different (such time in rotation waveform is significantly larger than that in the case when magnet is passing through the detector). This is also explainable because the polarity of the sensors' outputs changes as soon as the tracer magnet passes through the plane of the necklace, which happens relatively fast.

Obviously, the magnitude of the derivative waveform and the distance between its two extreme values are both dependant on how fast the patient rotates. Therefore, a combination of both values can provide a better criterion to remove the effect of earth magnetic field when the patient rotates. This part of the algorithm can be described as follows.

ÄT Threshold: ÄT is the time interval between the positive and negative peaks of the sensor outputs. These peaks usually occur around the time when the magnet passes the sensors plane and represents how fast the magnetic field changes its polarity. ÄT from passage of a magnet through the detector by ingestion is much smaller than ÄT from patient's normal rotations. Therefore, by setting an upper limit for ÄT, most rotations can be eliminated.
|dV/dt| Amplitude Threshold: This threshold acts as reinforcement criteria to reject fast rotations with small ÄT. Since the earth magnetic field similarly affects all sensors, in the case of a fast rotation, |dV/dt| response of all sensors of the same type will be large and above a certain threshold. In contrast, in a real ingestion event, at least one sensor output will have |dV/dt| below the threshold. With these additional thresholds, cases of either fast rotation with high dV/dt values or slow rotation with large ÄT values can be rejected.

The wireless handshaking protocol is preferably used to make sure that the “dose ingestion event” flag is properly transferred and data flow is achieved with a minimum error rate. The wireless communications have two operating modes: Normal mode and Program mode.

In the Normal mode: Transceiver 1 on detector side sends “0xAAAA” as a notification flag when a “dose ingestion event” is detected and Transceiver 2 on PC/PDA data delivery side answers with a “0xCCCC”. Transceiver 1 will send the flag again if it does not receive the correct response in a designated time period. Otherwise, the codes continue their normal operation. If Transceiver 1 still does not receive the correct response, it will repeat sending and listening for four more times before a red light is turned on for alarming the patient about the loss of connection. In this case, the necklace can continue detection but the PC/PDA might not be able to receive detection notification flags. Therefore the time and dose ingestion information will not be recorded, until the connection is resumed.

In Program mode every time the system starts up, it is running with its default threshold parameters. As soon as a qualified user finishes the parameter setting in LabVIEW GUI program and hits the “SET! !” button, PC/PDA will send a Program byte package (0x55+Parameter) to Transceiver 2. As a result, Transceiver 2 switches to Program mode and waits for the notification flag of the “dose ingestion event” from Transceiver 1 to start the programming. The working parameters including sensors' resolution, sampling rate and thresholds can be received by Transceiver 1 and updated in its memory, once there is an event coming up to trigger the wireless communication between the two transceivers. When programming is completed, the necklace starts the event detection algorithm based on the new set of parameters and Transceiver 2 switches back to its normal operating mode, listening for a “dose ingestion event” notification flag.

Graphical User Interface (GUI): After receiving the “ingestion event” from the transceiver through RS-232, the GUI time and date stamps the event and saves it in a spreadsheet file. The GUI also shows ten most recent events. Preferably, all the threshold parameters, which are used in the signal processing algorithm, can be adjusted and calibrated through the GUI screen and wirelessly updated on the detection device. This portion of the GUI, however, will only be accessible to the doctor or professional caregivers in the most preferred embodiment. Future versions of the GUI could also include schedule entry points to be programmed by the doctors in order to remind the patients to take their medications. If the patient misses a dose, he or she will be notified by the DCM system with a blinking LED or an audible alarm from the necklace.

### Example 1

The system described above in relation to FIG. 5 is used by a patient over a one month test regimen requiring ingestion of the medication of FIG. 1 on a regular basis every 8 hours. In addition, the patient keeps an accurate manual log showing the time and date of ingestion of the medication. A comparison of the ingestion event data logged using the instant invention with the manual log shows a correct ingestion event for the instant invention of 94.4%.

### Example 2

The system described above in relation to FIG. 5 is tested by passing the capsule shown in FIG. 1, 100 times on various locations near but outside of the necklace shown in FIG. 5. The data logged using the instant invention shows a 6% erroneous ingestion event detection rate.

### CONCLUSION

While the instant invention has been described above according to its preferred embodiments, it can be modified within the spirit and scope of this disclosure. This application is therefore intended to cover any variations, uses, or adaptations of the instant invention using the general principles disclosed herein. Further, the instant application is intended to cover such departures from the present disclosure as to come within the known or customary practice in the art to which this invention pertains and which fall within the limits of the following claims.

1. A method to measure compliance with a pharmaceutical regimen, comprising the steps of: (a) ingesting a dose of a medication into the gastrointestinal tract of a person, the dose of medication comprising a drug formulation and a permanent magnet; (b) as a result of the ingestion of step (a), detecting passage of the permanent magnet past at least two magnetic field sensors positioned apart from each other and adjacent to the gastrointestinal tract; and (c) measuring compliance with the pharmaceutical regimen by way of the detection of step (b).

2. The method of step 1, wherein step (b) comprises the generation of a time and date stamped data point is stored for use in step (c).

3. The method of step 2, wherein the time and date stamped data point is stored in digital form.

4. The method of step 3, wherein the data point is stored in a personal data assistant device or a personal computer.

5. The method of claim 1, wherein the presence of the permanent magnet inside the gastrointestinal tract is detected using magneto-inductive sensors.

6. The method of claim 5, wherein three magneto-inductive sensors are positioned apart from each other on a necklace worn around the neck of the person.

7. Apparatus useful to measure compliance with a pharmaceutical regimen by detecting ingestion of a dose of medication comprising a drug formulation and a permanent magnet, comprising: at least two magnetic field sensors positioned apart from each other on a necklace, each magnetic field sensor being in electronic communication with a microprocessor for receiving signals from the magnetic field sensors to determine the passage of a permanent magnet between the magnetic field sensors.

8. The apparatus of claim 7 wherein three magnetic field sensors positioned apart from each other are incorporated into the necklace.

9. The apparatus of claim 8 wherein the three magnetic field sensors are positioned about equidistant from each other.

10. The apparatus of claim 7, further comprising a digital personal data assistant configured to receive a time and date stamped data point transmitted by a radio frequency transmitter in electrical communication with the microprocessor.

11. The method of claim 2, wherein the presence of the permanent magnet inside the gastrointestinal tract is detected using magneto-inductive sensors.

12. The method of claim 3, wherein the presence of the permanent magnet inside the gastrointestinal tract is detected using magneto-inductive sensors.

13. The method of claim 4, wherein the presence of the permanent magnet inside the gastrointestinal tract is detected using magneto-inductive sensors.

14. The apparatus of claim 8, further comprising a digital personal data assistant configured to receive a time and date stamped data point transmitted by a radio frequency transmitter in electrical communication with the microprocessor.

15. The apparatus of claim 9, further comprising a digital personal data assistant configured to receive a time and date stamped data point transmitted by a radio frequency transmitter in electrical communication with the microprocessor.
